(19) United States
(12) Patent Application Publication
(10) Pub. No.: US 2022/0364896 A1
(43) Pub. Date: Nov. 17, 2022

(54) SITTING TOILET TYPE UROFLOWMETER APPARATUS

(71) Applicant: SapienMed Co., Ltd, Seoul (KR)

(72) Inventor: Seung June OH, Seoul (KR)

(73) Assignee: SapienMed Co., Ltd, Seoul (KR)

(21) Appl. No.: 17/569,512

(22) Filed: Jan. 6, 2022

Related U.S. Application Data
(60) Provisional application No. 63/188,412, filed on May 13, 2021.

(51) Int. Cl. G01F 3/38; E03D 11/13; A61B 5/20

(52) U.S. Cl. CPC G01F 3/38; E03D 11/13; A61B 5/208

(57) ABSTRACT

The present disclosure relates to a sitting toilet type uroflowmeter apparatus. The sitting toilet type uroflowmeter apparatus according to the present disclosure includes a toilet main body having a water collecting tank; a seat part that is positioned above the toilet main body and that is rotatable; and a uroflow sensor that is positioned on a bottom surface of the water collecting tank and that measures a flux over time.

### FIELD

The present disclosure relates to a sitting toilet type uroflowmeter apparatus, and more particularly, to a sitting toilet provided with a sensor for measuring uroflow.

### BACKGROUND

Voiding is a physiological activity of excreting biological metabolic by-products in liquid form out of the body. Symptoms of voiding dysfunction are observed in patients with various voiding diseases, such as prostatic hyperplasia, neurogenic bladder, overactive bladder, incontinence, cystitis, urethral stricture, and acquired dysuria, etc. These symptoms of voiding dysfunction include slow urine stream, urine stream interruption, urine hesitancy, terminal dribbling, urine straining, increased daytime urine frequency, nocturia, voiding urgency, and urinary incontinence, etc. To diagnose voiding dysfunction, questionnaire, physical examination, residual void volume test, imaging test, endoscopy, urodynamic study, and uroflowmetry are performed.

Urodynamic study is a collective term for tests made up of several detailed items such as uroflowmetry, filling cystometry, pressure-flow study, urethral pressure profile, and urethral sphincter electromyography. These tests are conducted selectively for patients with dysuria and urine incontinence. Urodynamic study provides the most important information in determining the diagnoses and treatment methods of patients with dysuria and urine incontinence. Especially, urodynamic study is an essential test for patients with various voiding dysfunctions because it can objectively determine the physiological functions of the lower urinary tract, which cannot be identified through questionnaire, physical examination, radiographic examination, or endoscopy.

However, the process and technique of the urodynamic study are complicated, and the test equipment for the urodynamic study has multiple test modules installed, which are interconnected to one another, and thus the equipment has a large volume and heavy weight. Further, since the urodynamic study is a detailed examination, the test takes quite a long time, and requires a procedure for inserting a catheter through the patient's urethra to the bladder, and thus there is also the risk of infection. Therefore, since the urodynamic study cannot be performed on all the patients with voiding dysfunction, it is being selectively performed on the patients who need it.

Uroflowmetry is the most non-invasive detailed test among urodynamic studies. A patient voids in a funnel-shaped uroflowmetry collecting device. The flow of the urine that the patient passes is calculated in weight according to several principles and is continuously input into a personal computer, and ultimately, is expressed in a graph form in units of volume/time. That is, uroflowmetry can objectively schematize and indicate the subjective voiding pattern of the patient.

In addition, uroflowmetry is a screening test that is used most widely and at the earliest stage for diagnosing voiding dysfunction patients not only because it can be performed quickly, but also because it is non-invasive, and is cost-effective. That is, if an abnormality is identified in the uroflowmetry, an additional detailed examination is required. Further, it is also widely used to determine the therapeutic effect before and after a drug or surgical treatment in patients with dysuria. After the uroflowmetry, the results are interpreted along with the residual urine volume measured by ultrasound to determine the efficiency of voiding.

A standard uroflowmetry involves measuring the weight of the urine during a voiding process with load cell and accumulating flow rate signals, and then computing clinically important diagnostic parameters such as the maximum flow rate, average flow rate, voiding time and voided volume. In addition, it is also important to check the pattern of the voiding curve, and the presence of symptoms such as slow stream, interrupted urine, terminal dripping, hesitancy, etc. can be confirmed as objective findings by matching with the uroflowmetry.

There are various methods such as gravimetric, rotating disk and the like that can be used as the principle of measuring uroflow, but gravimetric method is most widely used. A uroflowmeter apparatus of the gravimetric method is configured in the form of placing a load cell on the floor and placing a collecting container made of plastic material on top of the load again. The subject person voids into a funnel-shaped collecting container from above the collecting container and then changes in the weight of urine is measured. Here, a problem occurs where the impact on the container, shaking and noise are added to the measurement.

A patient's voiding pattern is often mutated depending on various factors, such as the greatness or smallness of the voided volume, the patient's tension before the examination, and general overall physical condition, often resulting in limited use of the test results. Therefore, normally, the test is performed two or more times, so that the most proper result of voiding pattern can be selected and utilized in diagnosis. Sometimes, in order to obtain a reliable and representative test result, there may be cases where multiple times of uroflowmetry are required from even one patient.

A uroflowmeter apparatus is usually placed on the floor, and thus in reality there are some important operational problems. First, once the patient completes the uroflowmetry, medical staff must clean up the urine each time. That is, since uroflowmetry is a screening test, many patients are tested every day, and thus there is a sanitary problem in that it must be quickly and thoroughly emptied before the next patient is tested. In other words, this requires consumption of medical personnel. Second, disposable transparent plastic cups are mostly used as urine collecting containers, and since uroflowmetry is used as a screening test in many patients, this also causes environmental problems. Third, there is a problem that patients may unintentionally kick the uroflowmeter apparatus with their feet when approaching the apparatus installed on the floor. Especially, patients with weak uroflow tend to come closer to the test equipment in order not to spill urine out of the container. A weight sensor is attached to the uroflowmeter apparatus, so when a patient touches it, an artifect might occur, leading to an error in the test results. Fourth, since patients are instructed to void in front of an artificial device, there is a disadvantage that it is difficult to accurately reflect the patient's voiding pattern under their usual natural conditions. Voiding is a highly private and personal basic physiological activity, so it is greatly affected psychologically. Due to the special nature of this test, the patient cannot help but feel psychological atrophy and tension in the artificial environment of the hospital, such as where the uroflowmetry takes place.

The utilization of uroflowmetry in the diagnostic process for patients with voiding dysfunction is very high. However, various realistic problems that accompany the uroflowmetry as mentioned above need to be resolved. In reality, it is necessary to devise an apparatus that may be installed in actual toilets and that has the shape closest to the existing toilet type, and where even if it touches the patient's foot, an error does not occur in the recording signal. Further, such an apparatus should not require additional medical personnel for cleaning up, nor contain any consumables.

### SUMMARY

Therefore, a purpose of the present disclosure is to resolve the aforementioned problems of prior art, that is, to provide a sitting toilet type uroflowmeter apparatus that is not affected by the movement of a patient during the test and that allows easy clean up after the test.

Another purpose of the present disclosure is to provide a sitting toilet type uroflowmeter apparatus that can minimize the impact applied on a measuring container and the noise caused by the shaking during the voiding process.

A sitting toilet type uroflowmeter apparatus according to an embodiment of the present disclosure for achieving the aforementioned purposes may include a toilet main body 100 having a water collecting tank; a seat part 110 that is positioned above the toilet main body 100 and that is rotatable; and a uroflow sensor 120 that is positioned on a bottom surface of the water collecting tank and that measures a flux over time.

Desirably, the sitting toilet type uroflowmeter apparatus may further include a drain pipe 130 through which water pooled in the water collecting tank may be drained, and one end of the drain pipe 130 may communicate with one side at a lower end of the water collecting tank, and the other end of the drain pipe 130 may communicate with a sewage pipe, and at one end of the drain pipe 130, an outlet valve 140 may be formed for opening and closing a passage between the drain pipe 130 and the water collecting tank.

Desirably, the seat part 110 may have a pressure sensor 110s for sensing a pressure applied on the seat part 110, and the outlet valve 140 may be closed when the pressure sensor 110s senses the pressure.

Desirably, the uroflow sensor 120 may consist of a plurality of load cells 121, and the plurality of load cells 121 may be positioned on vertices of a regular polygon to form a symmetrical structure with one another, and a plurality of signals calculated by the plurality of load cells 121 may be summed to measure the flux over time.

Desirably, an elastic body 122 and a housing 123 that supports the elastic body 122 may be formed above the plurality of load cells 121.

The present disclosure may provide a sitting toilet type uroflowmeter apparatus that is not affected by the movement of a patient during the test and that allows easy clean up after the test.

The present disclosure may provide a sitting toilet type uroflowmeter apparatus that can minimize the impact applied on a measuring container and the noise caused by the shaking during the voiding process.

### BRIEF DESCRIPTION OF THE DRAWINGS

FIG. 1 is a view illustrating a configuration of a sitting toilet type uroflowmeter apparatus according to an embodiment of the present disclosure;

FIG. 2 is a view illustrating a configuration of a sitting toilet type uroflowmeter apparatus having a plurality of load cells according to another embodiment of the present disclosure;

FIG. 3 is a view illustrating a configuration of a uroflow sensor having an elastic body according to another embodiment of the present disclosure; and

FIG. 4 is a view for describing operations of a controller of a sitting toilet type uroflowmeter apparatus according to another embodiment of the present disclosure.

### DETAILED DESCRIPTION

Hereinbelow, some embodiments of the present disclosure will be described in detail through the exemplary drawings. In adding reference numerals to components of each drawing, it should be noted that even if the components are displayed on different drawings, like reference numerals are used for like components as much as possible.

Further, in describing the embodiments of the present disclosure, if it is determined that a specific description of a related well-known configuration or a function interrupts the understanding of the embodiments of the present disclosure, detailed description thereof will be omitted.

Further, in describing the components of the present disclosure, terms such as a first, a second, A, B, (a), (b) and the like may be used. Such terms are merely used to distinguish those components from other components, and such terms do not limit the nature, sequence or order of the corresponding components.

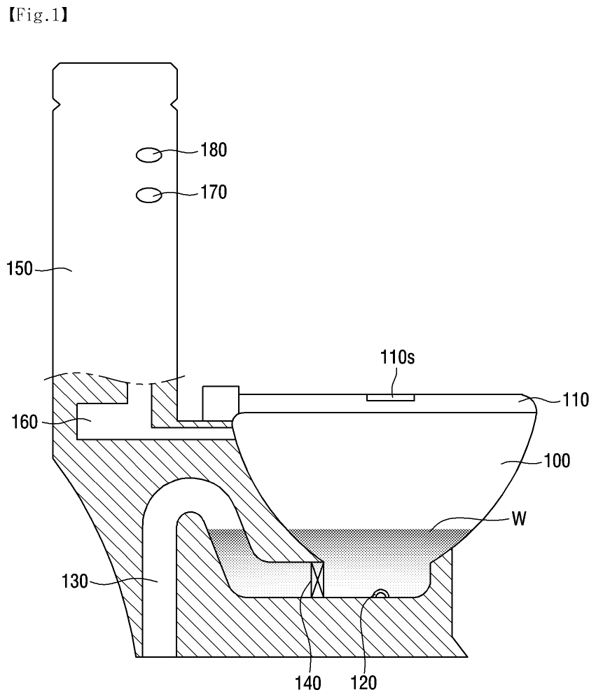

Hereinbelow, referring to FIGS. 1 to 4, a configuration and operations of a sitting toilet type uroflowmeter apparatus according to an embodiment of the present disclosure will be described.

FIG. 1 is a view illustrating the configuration of a sitting toilet type uroflowmeter apparatus according to an embodiment of the present disclosure (hereinafter referred to as “the present uroflowmeter apparatus”).

Referring to FIG. 1, the present uroflowmeter apparatus may include a toilet main body 100 having a water collecting tank, a seat part 110 positioned above the toilet main body 100 and that is rotatable, and/or a uroflow sensor 120 that is positioned on a bottom surface of the water collecting tank to measure the flux over time.

The uroflow sensor 120 measures the weight of the fluid initially pooled in the water collectiing tank, and then measures the weight of fluid being increased by the voiding of a subject person being tested, over time. The uroflow sensor 120 measures the weight of fluid over time, and a controller 200 calculates the volume of the fluid over time based on the weight signal measured by the uroflow sensor 120.

Specifically, when urine is introduced into the water collecting tank by the voiding of the subject person being tested, the weight of the fluid being pooled in the water collecting tank increases according to the volume of the urine being introduced. Here, the weight (W) of the fluid is the product of the mass (m) of the fluid and the gravitational acceleration (g), and the mass of the fluid can be obtained by multiplying the volume (V) and the density (p) of the fluid (Equation (1)). Moreover, the change in the volume of the fluid being introduced over time is referred to as the flow rate (F), and the change in the volume of the urine being introduced when the subject person voids becomes the urine flow rate (flux per hour).

Therefore, the urine flow rate is defined as the time differential function of the urine volume as in Equation (2), and can be obtained by mathematically differentiating V in Equation (1). This indicates that by continuously measuring the weight of the urine being filled in the water collecting tank, a urine flow rate signal can be calculated. However, in the present specification, it is presumed that water and urine have the same mass and density.

According to another embodiment of the present disclosure, the

$$F = \frac{dV}{dt} = \frac{1}{ρ g} \cdot \frac{dW}{dt} \qquad ( 2 )$$

present uroflowmeter apparatus may further include a drain pipe 130 through which the water pooled in the water collecting tank may be drained and/or an outlet valve 140 for opening and closing a passage between the drain pipe 130 and the water collecting tank.

One end of the drain pipe 130 communicates with one side at a lower end of the water collecting tank, and the other end of the drain pipe 130 communicates with a sewage pipe (not illustrated), and at one end of the drain pipe 130, the outlet valve 140 for opening and closing the passage between the drain pipe 130 and the water collecting tank is formed.

The drain pipe 130 is formed in an inverted U-shape by a trap method, whereby the fluid being pooled in the water collecting tank maintains a certain amount. That is, when the subject person voids in the water collecting tank, the fluid increased in the water collecting tank is drained to the sewage pipe through the drain pipe 130 by the Siphon principle, and thus the amount of fluid pooled in the water collecting tank is always kept constant.

Due to this Siphon principle, existing sitting toilets are problematic in that they do not allow to measure the changes in urine volume according to the voiding of the subject person, and thus in order to resolve this problem, the present disclosure included the outlet valve 140 between the drain pipe 130 and the water collecting tank.

According to the present disclosure, before the subject person voids, the outlet valve 140 is closed, so that the fluid pooled in the water collecting tank may be continuously pooled in the water collecting tank instead of flowing to the drain pipe 130. Thus, the flux over time of the fluid that increases due to the voiding of the subject person can be measured by the uroflow sensor 120. In addition, when the subject person finishes voiding, the outlet valve 140 is opened, so that the fluid pooled in the water collecting tank may be drained to the sewage pipe through the drain pipe 130. Here, as the water pooled in a water tank 150 is introduced into the water collecting tank through a water supply pipe 160, together with the fluid that was pooled beforehand, urine is drained by a greater pressure to the sewage pipe through the drain pipe 130. This draining process is similar to that of normal toilets of prior art.

According to another embodiment of the present disclosure, the seat part 110 provided in the present uroflowmeter apparatus has a pressure sensor 110s for sensing a pressuring being applied to the seat part 110, and the outlet valve 140 is closed when the pressure sensor 110s senses the pressure.

That is, when the pressure is sensed by the pressure sensor 110s, it may be recognized that the subject person will void soon, and when an electrical signal is received from the pressure sensor 110s, the controller 200 closes the outlet valve 140 and initiates operations of the uroflow sensor 120.

Further, for cases where a male subject person rotates the seat part 110 and then voids, or where a female subject person voids while keeping her buttocks apart from the seat part 110, the pressure sensor 110s may sense the rotation of the seat part 110. Here, any method for sensing the rotation may be used, but the pressure sensor 110s may sense the pressure at a rotation axis (not illustrated) of the seat part 110, or the pressure in the process where the seat part 110 touches the water tank 150.

That is, the pressure at the rotation axis, the pressure when touching the water tank 150, and/or the weight pressure of the subject person may be sensed by the pressure sensor 110s, and then the controller 200 may activate a preparation process for pooling urine.

Otherwise, together with such a pressure sensor 110s or separately from the pressure sensor 110s, the subject person may manually use a pooling switch 170 to allow the controller 200 to perform the preparation process for pooling urine. That is, when the subject person turns on the pooling switch 170, the outlet valve 150 may be closed by the controller 200, and when the subject person turns off the pooling switch 170, the outlet valve 150 may be opened. Such a pooling switch 170 may have the form of a button, lever and the like.

Otherwise, the present uroflowmeter apparatus may further include a flushing switch 180. When the subject person finishes voiding and operates the flushing switch 180, the outlet valve 150 may be opened by the controller 200, and at the same time, as the water pooled in the water tank 150 is introduced into the water collecting tank through the water supply pipe 160, together with the fluid pooled beforehand, the urine may be drained to the sewage pipe through the drain pipe 130. Such a flushing switch 180 may have the form of a button and lever, etc.

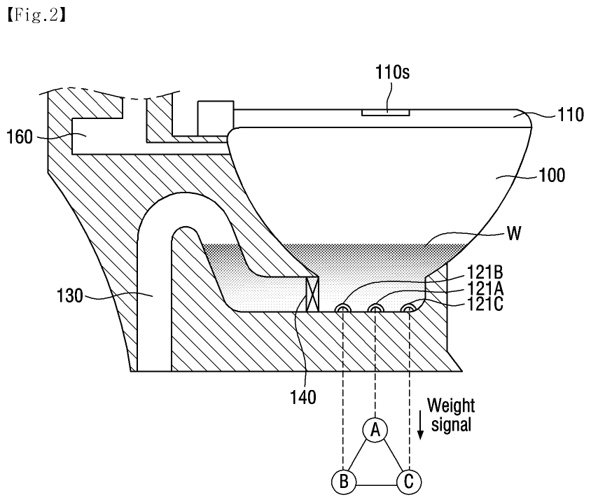

FIG. 2 is a view illustrating the configuration of a sitting toilet type uroflowmeter apparatus having a plurality of load cells according to another embodiment of the present disclosure.

Referring to FIG. 2, the uroflow sensor 120 provided on the bottom surface of the water collecting tank consists of a plurality of load cells 121A, 121B, 121C, and the plurality of load cells 121A, 121B, 121C are positioned on vertices of a regular polygon, forming a symmetrical structure to one another. In addition, the controller 200 sums up the plurality of signals calculated by the plurality of load cells 121A, 121B, 121C, to measure the flux over time. Specifically, a weight signal regarding the weight that the plurality of load cells 121A, 121B, 121C sensed is transmitted to the controller 200, and the controller 200 sums up the weight signal transmitted from each load cell 121A, 121B, 121C, and converts them into volume signals to measure the flux over time. A detailed description of the controller 200 will be made later.

The urine being introduced into the water collecting tank during the voiding process may be introduced through a wall surface of the water collecting tank or may be introduced while directly impacting the surface of the fluid pooled in the water collecting tank. In this process, since the uroflow sensor 120 measures the weight of fluid per hour, at the moment when the urine impacts the pooled fluid, the weight is measured to be greater than the actual weight, and thus there is a problem that the weight of fluid per hour cannot be measured accurately.

In order to resolve this problem, on the bottom surface of the water collecting tank, the present disclosure has a plurality of load cells 121A, 121B, 121C that sense the weight of fluid, and the plurality of load cells 121A, 121B, 121C are positioned on vertices of a regular polygon, forming a symmetrical structure to one another. When the load cells 121A, 121B, 121C are configured as described above, each load cell 121A, 121B, 121C senses the weight that is equal to the weight (W) of the total fluid pooled in the water collecting tank divided by the number of the load cells. Here, a noise that is caused by an impact is intervened in the weight sensed by each load cell 121A, 121B, 121C, and each noise that is intervened herein corresponds to a random noise having an average value of 0, and thus the sum of the noise becomes close to 0. Therefore, by summing the plurality of weight signals sensed by each load cell 121A, 121B, 121C, it is possible to minimize the noise caused by the impact. That is, by summing and averaging the weight signals measured from the plurality of symmetrical positions, it is possible to obtain a urine flow rate signal from which noise has been removed.

For example, in a case where three load cells 121A, 121B, 121C are positioned on three vertices A, B, C of a equilateral triangle to form a symmetrical structure on the bottom surface of the water collecting tank, the weight that each load cell senses WA, WB, WC is as in Equation (3) below, and the weight of the total fluid obtained by summing and averaging each weight signal is as in Equation (4) below. In addition, when Equation (4) is converted into urine flux signal that is a volume unit, it is the same as Equation (5) below. In the Equations below, eA, eB, eC represent the noise intervened in each load cell.

$${W}_{A} = \frac{W}{3} + {e}_{A} , {W}_{B} = \frac{W}{3} + {e}_{B} , {W}_{C} = \frac{W}{3} + {e}_{C} \qquad ( 3 )$$

$${W}_{MEAN} = {W}_{A} + {W}_{B} + {W}_{C} = W if {e}_{A} + {e}_{B} + {e}_{C} = 0 \qquad ( 4 )$$

When comparing the urine flux signal measured in the

V_{MEAN}=V_{A}+V_{B}+V_{C}=V

individual load cell 121A or 121B or 121C with the summed and averaged urine flux signal measured in the three load cells 121A, 121B, 121C according to an embodiment of the present disclosure, in the urine flux signal measured in the individual load cell 121A or 121B or 121C, a large noise was intervened at a time point of 30 seconds after the voiding started and at a time point of 50 seconds after the voiding started, whereas in the summed and averaged urine flux signal measured in the three load cells 121A, 121B, 121C according to an embodiment of the present disclosure, a small noise was intervened at a time point of 30 seconds after the voiding started and at a time point of 50 seconds after the voiding, and there was no large scale registration.

Thus, it was shown that at the time point of 30 seconds and 50 seconds after the time point when the voiding started, the voiding volume decreased, and accordingly, the urine failed to form a stream and instead the dripping urine impacted the pooled fluid, resulting in a noise intervening in the urine flux signal, and test results showed that the noise caused by such an impact can be significantly offset by the summing and averaging method of the present disclosure.

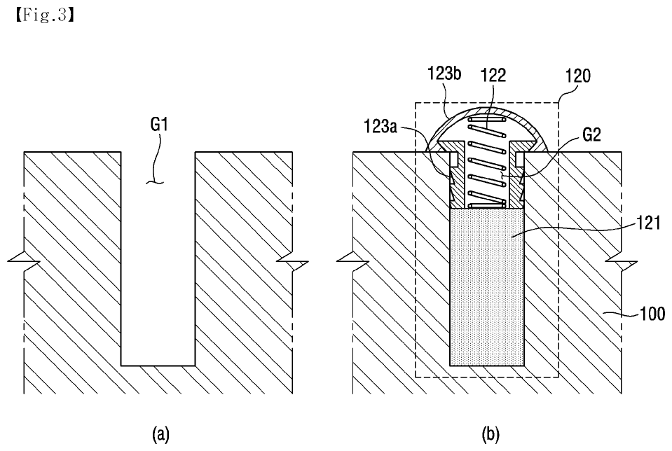

FIG. 3 is a view illustrating a configuration of a uroflow sensor having an elastic body according to another embodiment of the present disclosure.

Referring to FIG. 3, the uroflow sensor 120 provided on the bottom surface of the water collecting tank inside the toilet main body 100 of the uroflowmeter apparatus of the present disclosure may include a load cell 121 for sensing weight, an elastic body 122 provided above the load cell 121, and/or housings 123a, 123b for supporting the elastic body 122.

Such a structure of the uroflow sensor 120 is to cushion the impact that urine can have on the fluid pooled in the water collecting tank during the voiding process, and thereby attenuate the noise caused by the impact, and accurately sense the weight of the fluid itself only.

Specifically, on the bottom surface of the water collecting tank, a groove G1 is formed so that the uroflow sensor 120 can be inserted and immobilized, and in the formed groove G1, the uroflow sensor 120 is inserted and installed. In the groove G1, the load cell 121 for sensing the weight of the fluid is inserted and installed, and above the load cell 121, the support housing 123a is inserted and installed. Here, a wedge-shaped locking projection is formed on an outer periphery of an insertion part of the support housing 123a. Thereafter, in a central groove G2 of the support housing 123a, the elastic body 122 is inserted, and on an opening part of the groove G2, a cover housing 123b made of a soft material and having a convex circular shape is formed.

The elastic body 122 as a cushioning means inside such a groove G2 may be formed inside the cover housing 123b made of a soft material such that it has a caliber that maintains a certain gap with an inner periphery of the groove G2. Thus, the impact force generated by the urine being voiding is absorbed by the cover housing 123b and the elastic body 122, and thus offset. That is, since the caliber of the elastic body 122 is smaller than the inner diameter of the groove G2, when the impact force is transmitted, the elastic body 122 is easily contracted to effectively offset the impact force.

However, the caliber of the elastic body 122 is not smaller than the inner diameter of the groove G2 throughout the entire length. A lower caliber of the elastic body 122 should have a size large enough to fit tightly to the groove G2, while a middle part or upper part of the elastic body 122 is smaller than the inner diameter of the groove G2, if it were not to fall out unless it is pulled out by applying artificial force, so that the cover housing 123b and the support housing 123a can maintain a state where they are coupled to each other even without additional immobilizing means, and when impacted, the elastic body 122 can be easily contracted, thereby doubling the effect of offsetting the impact force. Here, the elastic body 122 may be an object having the form of a rod made of an elastic material, or a spring.

Otherwise, in another embodiment, as a cushioning means inside the groove G1 of the support housing 123a, instead of projecting the elastic body 122 in the cover housing 123b, if compressed air is put into the groove G1 and the inside of the cover housing 123b and the inside of the groove G1 are sealed, when the impact force is transmitted, due to the compressed air, the housing 123 can absorb and offset the impact force as much as possible, and thereby reduce the noise caused by the temporary impact force.

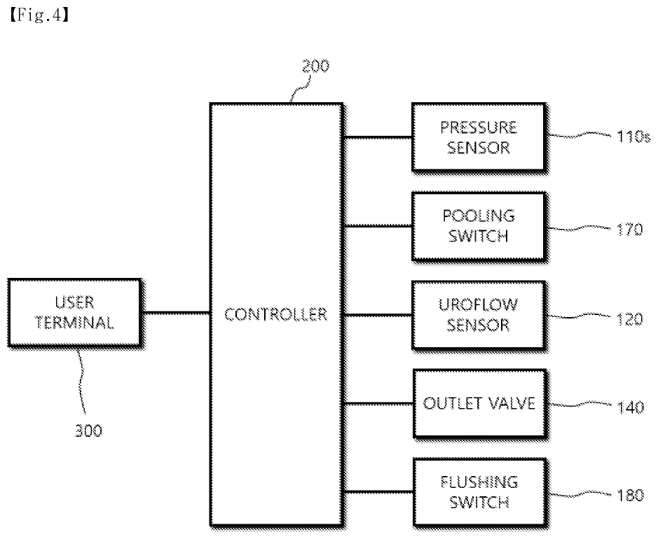

FIG. 4 is a view for describing operations of the controller of a sitting toilet type uroflowmeter apparatus according to an embodiment of the present disclosure.

Referring to FIG. 4, the uroflowmeter apparatus of the present disclosure may be implemented as a system including the controller 200 and/or a user terminal 300.

The controller 300 may receive an electrical signal from a pressure sensor 110s, a pooling switch 170, and/or a flushing switch 180, and control operations of the uroflow sensor 120 and/or outlet valve 140 according to the received signal. Moreover, the controller 200 may convert a weight signal sensed and transmitted by the uroflow sensor 120 into a volume signal, or sum and average the weight signals received from the plurality of load cells, and then convert the summed and averaged weight signal of the fluid into a volume signal. Such a converted volume signal according to time may correspond to a urine flow rate signal, and this urine flow rate signal may be transmitted to the user terminal 300.

The user terminal 300 may correspond to a memory device, a PC, a smart phone, a wearable device, a display device, and the like, and the controller 200 and the user terminal 300 may be connected through various wired/wireless communication networks.

The embodiments disclosed in the present specification belong to the same technical field and components constituting one embodiment may be combined with components constituting another embodiment to constitute a new embodiment.

The protection scope of the present disclosure is not limited to the description and expressions of the embodiments explicitly described above. In addition, it is added once again that the protection scope of the present disclosure cannot be limited due to obvious changes or substitutions in the technical field to which the present invention pertains.

What is claimed is:

1. A sitting toilet type uroflowmeter apparatus comprising:
a toilet main body having a water collecting tank;
a seat part that is positioned above the toilet main body and that is rotatable; and
a uroflow sensor that is positioned on a bottom surface of the water collecting tank and that measures a weight of fluid pooled in the water collecting tank over time.

2. The sitting toilet type uroflowmeter apparatus according to claim 1,
further comprising a drain pipe through which water pooled in the water collecting tank may be drained, and
one end of the drain pipe communicates with one side at a lower end of the water collecting tank, and the other end of the drain pipe communicates with a sewage pipe, and at one end of the drain pipe, an outlet valve is formed for opening and closing a passage between the drain pipe and the water collecting tank.

3. The sitting toilet type uroflowmeter apparatus according to claim 2,
wherein the seat part has a pressure sensor for sensing a pressure applied on the seat part, and the outlet valve is closed when the pressure sensor senses the pressure.

4. The sitting toilet type uroflowmeter apparatus according to claim 1,
wherein the uroflow sensor consists of a plurality of load cells, and the plurality of load cells are positioned on vertices of a regular polygon to form a symmetrical structure with one another, and a plurality of signals calculated by the plurality of load cells are summed to measure the flux over time.

5. The sitting toilet type uroflowmeter apparatus according to claim 4,
wherein an elastic body and a housing that supports the elastic body are formed above the plurality of load cells.
